SEQUENCE LISTING (2 sequences)

SEQ ID NO: 1 - DNA, 990 bp, Medicago truncatula
  source 1..990: mol_type = other DNA
atgagtatgttaggtcttagagacctagttctcatagctccatcaccttcttctcttcaacatcatcatcaacaaaatcaaaatcaaaaccaaaaccaaaaccaacccatttcacatgatcacaattcaaaccactctttaccttcatcagcttcattaagtgttggttttggcatttttccacttctcactgctacaccttgcatgccacaacaacaatctcaaaacaatgaagttcaagaaaatcctagtaacaataacaatttttggaatcttaggatgtgtcctgaagtagtgaatcttccaaaaaaaggagtgatcactgaagaagagaatcatggaaaaattgctatgatggagagtgaagaaaatggtgtgtatggttctgagtatagagtgtgtcaagattgtggtaatagggctaagaaagattgtgtgtttagaagatgtaggacttgttgtaagggacgtggttatgattgtagtactcatttgaagagtacttggattccttcgacacgtcgtcgagaacgggaggtggagatgtttgccggtggtggtgatggtgagggttgttctggtgttaagagacaaaaaggtttattagggtcatcacaaaatgctgctgctacttctcatagctctaactctaatggtactacaccaaagagttttgctactagctcctgtcaccaagatgcaggtttcaaagagggtttaccaggtcatgttaatgcaccagctgtattcaggtgccatagagtaactgctattggcaatggtgaagatgaatttgcttacttggccacagttcatattagtggccatgttttcaaagggtttctctatgatcatggtgttgatgggaaaaatccaatgccttgtgtttcagaacttcaattgggaaataattgtagtggaaaaaatggtgaatgttcttctgcaattggtgttcccaacaataatgcttaccctgcttctgctagctag

SEQ ID NO: 2 - DNA, 3603 bp, Medicago truncatula
  source 1..3603: mol_type = other DNA
atggcaaaaaacatagacaaaacaagttctgattctgaaaattctttatgttcatcaagttcaagccctccaatgagtaataagtataggtacatgttcaatagactattcaggtttgccaattcatggaagaaaagtaactcaccaagaggtagaagaatcttccatagggatgttgagaaagaagaatttcaatatgctagtagtcactgtctttcatcctactacagtgtttttgtggttagattagcaatcatggtaatgttagccatcttgatagggctattgaccatattaacatggcatttcacaaagatttatacgaaaaaatcattgagtagtttggcatatggtctgcgttatgaacttctgcaacgtccgattttgaggatgtggaatattttgaattctacttctgaaattacaacagctcaggtcaagttgtctcaatatgttatcagacgttatagcaaccctgcatctcaagctgagcaagttgagctatatgaagcaatgagggcagtaacatggtcactatttgctagtagaaaagcactaaactcaataacaatcaattacaagaatggatttgttcaagcattccatagagatctaaaagataacaacacattttacatctactcagatctttctaattactccatggttgcaacaacctcaaacatgctcaagtccatttcaactcatcaagcttgggatgataaaacacttcatggaaacttctcagcaatttggtatagagaaccacttgatcctgtcacaggcgaaaaaatcggaaaagcaatgaaaattgcaccagaagatttgatcaacatagcaggactttcacaagttcctgatggtgtagctacatggcatgtagcagttagtaaatttacagattcacctttgctttcagcagcattacctgtttgggattcatcgaataaaagtattatggctgttgtcggtgtaacgactgcattttatagtgttggacagttaatgagagagttagttgagatgcatagtggtcacatgtatttgacttctcaacaaggctatttacttgcaacttctacaagtgcacctttgcttacaaattcaactaagccgcctccaaagcttaagatggcggtggattgtgaagatgaaataataagacttggagccgagtggctgcagagaacttatggaaatcattttcctaattcaactcatgaggttcatgttgagaatgctaagcttgggaatcagcaatattacatcgactcgttctacctaaacttgaagagacttcctttggtaggcgtaatcatcataccaagaaagtatatcatgggacaggtggatgagagatctttcaagacattggttattctgatatctgcatctttatgcatttttgtcattggatgtgtttgcattttgatattgacaaatggagtgtcaaaggaaatgaatctgagagcagaactgataagtcaacttgaagcaagaaggaaggcagaggcatcaagcaactataaaagtcaattccttgcaaacatgagtcatgaactaaggacacctatggcagcagtaattggattgcttgacattcttatatctgatgattgtctcacaaatgaacagtattcaacagttactcaaataagaaaatgttcaacagctctactccgacttcttaacaacatattggatctgagcaaggtggagtctggaaaacttgtactagaagatgcagaattcgatctgggacgcgaacttgaagggcttgtagatatgttttctgttcagtgcattaatcacaatgtagagactgttttagacctttctgatgatatgccaaaattagttagaggtgactctggcagggtggttcaagtatttgcaaatttgatcaacaattcaattaagtttactacatctggtcatgttattctacgaggatggtgtgaatatccaaactcttgcaacgatagtccgaatttttatcttgatcagaagaaatcaagaaatttacataagagcaaagagaagccaaatgcaagtcatgcaaagagaatatctatgagagataacaaagtgatactttggtttgaagttgaggacacaggctgtggtattgatccaagcaaatgggattctgtgtttgaaagctttgagcaagctgatccatcaactactcgactgcatggagggactggtcttggtctttgcattgttaggaacttggttaataagatgggtggagaaatcaaggttaccaaaaaggagggtcaaggaacattgatgagattatgtttgcttcttagtgcaccagtggatgtcacagagcaacatgctttaaatttgacagacaatggcttagtagtattgcttgcactacaaggaaacatgggtagaataataacatcaaagtggttaaccaaaaaaggggtgtgcacaatggaagcatctgaatggaatggactaacacaaattttgagggaactctttcatgcaagaagttcaatccataacagtaacaactttgatgcacactatccaataaatcttgaaggatttaagtctaaactactaagcatcaaagacatgaggaatccaatttttgtcattgttgttgacattggtttgcttgacttaagcacagatatatggaaagaacaacttaacttccttcacaagtattttggaagagcgaaatttgtgtggatgctgaatcatgacacatcaaataccataaagacggagcttcgtcggaaaggacacttattaatggttaacaaacctctttacaaagcaaaaatgattcatattttggaagctgttataaaagagagaaatcatgagttgcaaaagaaaaacatgaaagagggtgatttgcatgagtttcttgagattgattctactcattttgatgctgctactagctctgatgattctgacatatctgaaattagtgaaaaaccaattgtaaagttaaccaatgaaaatgaacatttggaagaaaataattcaaggaaagaagaatcttgttcctctgaagagaagaaagctaaagaaacaaactttgcaacacaaggttatgatagggtgacaaggacaagaaaagaagtgaatgataagaaatcacttgaggggttaaggattttgcttgcagaagatacaccagtaattcaaagggttgcaacaataatgcttgaaaaaatgggagctgttgttgttgctgtaggtgatggacaacaagcagtggatgctcttaattatatggcaagtgctgaagattgtagaagagagtctctccagaaggacagaaacacaagatcacagacagagattttaacatgccatcaatatgatttgatcctaatggattgtcagatgccaaagatggatggatatgaggcaacaaaagaaataaggaaatcagaggaaggaacaggaaaacacattccaattgttgctctaacagcccatgcaatgtcttgtgatgaagccaaatgccttaaggttggcatggatgcttacttaacaaagccaattgacttcaagcttatggtctcaaccattctctcactcactaaaccaacaaactag

(19) United States
(12) Patent Application Publication
(10) Pub. No.: US 2024/0384283 A1
(43) Pub. Date: Nov. 21, 2024

(54) TRANSGENIC PLANT AND METHODS OF STIMULATING SPONTANEOUS NODULE FORMATION IN NON-LEGUME PLANTS

(71) Applicants: UNIVERSITY OF FLORIDA RESEARCH FOUNDATION, INCORPORATED, Gainesville, FL (US); WISCONSIN ALUMNI RESEARCH FOUNDATION, Madison, WI (US)

(72) Inventors: Matias Kirst, Gainesville, FL (US); Jean-Michel Ané, Madison, WI (US); Daniel Conde Rodriguez, Gainesville, FL (US); Wendell Pereira, Gainesville, FL (US); Paolo Maria Triozzi, Gainesville, FL (US); Christopher Dervinis, Gainesville, FL (US); Kelly Mayrink Balmant, Gainesville, FL (US)

(21) Appl. No.: 18/692,979

(22) PCT Filed: Sep. 23, 2022
(86) PCT No.: PCT/US22/76942

§ 371 Date: Mar. 18, 2024

Related U.S. Application Data
(60) Provisional application No. 63/248,125, filed on Sep. 24, 2021.

(51) Int. Cl. C12N 15/82

(52) U.S. Cl. CPC C12N 15/8241; C12N 15/8227

(57) ABSTRACT

The disclosure relates to genetically modified plants and methods of producing a non-legume plant capable of spontaneous nodule formation.

### CROSS-REFERENCE TO RELATED APPLICATIONS

The present application claims the benefit of priority to U.S. Provisional Application No. 63/248,125 filed Sep. 24, 2021, the disclosure of which is incorporated by reference in its entirety.

### STATEMENT OF GOVERNMENT SUPPORT

This invention was made with government support under DE-SC0018247 awarded by United States Department of Energy. The government has certain rights in the invention.

### INCORPORATION BY REFERENCE OF THE SEQUENCE LISTING

This application contains, as a separate part of disclosure, a Sequence Listing in computer-readable form (filename: 57027_Seqlisting.xml; Size: 7,231 bytes: Created: Sep. 1, 2022) which is incorporated by reference herein in its entirety.

### FIELD

The disclosure relates to genetically modified plants and methods of producing a non-legume plant capable of spontaneous nodule formation.

### BACKGROUND

The acquisition of mineral nutrients is one of the major challenges for plant survival. In particular, the macronutrient nitrogen (N) is one of the most limiting plant growth factors. Despite being the most common chemical component of Earth's atmosphere, plants cannot access N directly but must obtain it from the soil as nitrate, ammonium, or amino acids. Alternatively, a clade of angiosperms has overcome N limitations via interactions with symbiotic bacteria. In this interaction, the bacteria invade the plant root and are contained in intracellular compartments within a specialized organ, the nodule. In the nodule, dinitrogen (N₂) is converted into ammonia, which is usable by the plant. Engineering nodule formation in plants that are not capable of nodulation would create crops that are capable of nitrogen fixation.

### SUMMARY

In one aspect, described herein is a transgenic plant comprising at least one nucleotide sequence that is at least 85% identical to SEQ ID NO: 1 and/or SEQ ID NO: 2.

In another aspect, described herein is a method of producing a non-legume plant capable of spontaneous nodule formation comprising introducing one or more nucleotide sequences that are at least 85% identical to SEQ ID NO: 1 and/or SEQ ID NO: 2 into a cell of the plant.

In another aspect, described herein is a method of stimulating spontaneous nodule formation in a non-legume plant comprising introducing one or more nucleotide sequences that are at least 85% identical to SEQ ID NO: 1 and/or SEQ ID NO: 2 into the genome of the plant.

A seed produced by a plant described herein is also contemplated.

### BRIEF DESCRIPTION OF THE FIGURES

FIGS. 1A and 1B show functional characterization of MtLRP1 and MtHK1. FIG. 1A relates to composite plants inoculated with E. meliloti constitutively expressing lacZ. Detection of lacZ activity was used to monitor the successful nodule infection. Positive events of the transformation are identified as the ones emitting the red fluorescence of the DsRED protein. FIG. 1B relates to nodule number of composite plants that carried the empty vector as a control (white boxes) or LRP1 and HK1 RNAi constructs (grey boxes). For each box-and-whiskers plot: the center black line represents the median; ‘+’ represents the mean; the box extends from the 25^{th} to 75^{th} percentiles; the whiskers are drawn down to the 10^{th} percentile and up to the 90^{th}. Different lowercase letters indicate significant differences. P-values determined by one-way analysis of variance (ANOVA) followed by Tukey's multiple comparison test.

FIG. 2 is an image of the nodule structure in Populus plants. Roots transformed with MtHK1 under control of the pericycle promoter display nodule structures, in the absence of phytohormones.

### DETAILED DESCRIPTION

Nodule development begins with cell division of the pericycle cells—this step triggers the developmental process that results in nodule formation. The present disclosure is based, at least in part, on the discovery of genetic regulators of the early differentiation of pericycle cells during nodule formation (e.g., hexokinase 1 (HK1) and lateral root primordium (LRP1)). As shown in the Examples, expression of HK1 or LRP1 induced the formation of nodule structures in the non-nodulating species Populus trichocarpa, indicating that HK1 and LRP1 (either alone or in combination) are necessary and sufficient to trigger the development of nodules in species that lack this capability.

In one aspect, described herein is a transgenic plant comprising at least one nucleotide sequence that is at least 85% identical to SEQ ID NO: 1 or SEQ ID NO: 2.

In some embodiments, the transgenic plant comprises a nucleotide sequence that is at least 85% (e.g., at least 85%, 86%, 87%, 88%, 89%, 90%, 91%, 92%, 93%, 94%, 95%, 96%, 97%, 98%, 99% or more) identical to the nucleotide sequence set forth in SEQ ID NO: 1. In some embodiments, the transformed plant comprises the nucleotide sequence set forth in SEQ ID NO: 1.

In some embodiments, the transgenic plant comprises a nucleotide sequence that is at least 85% (e.g., at least 85%, 86%, 87%, 88%, 89%, 90%, 91%, 92%, 93%, 94%, 95%, 96%, 97%, 98%, 99% or more) identical to the nucleotide sequence set forth in SEQ ID NO: 2. In some embodiments, the transgenic plant comprises the nucleotide sequence set forth in SEQ ID NO: 2.

In some embodiments, the transgenic plant comprises the nucleotide sequences set forth in SEQ ID NO: 1 and SEQ ID NO: 2.

In some embodiments, the nucleotide sequence is expressed in a pericycle cell of the plant. In some embodiments, the nucleotide sequence is expressed in a cortex cell of the plant.

As used herein, the term “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

In some embodiments, the nucleic acid molecules described herein can be “optimized” for enhanced expression in plants of interest (see, for example, WO 91/16432).

In this manner, the open reading frames in genes or gene fragments can be synthesized utilizing plant-preferred codons (see, for example, Campbell & Gowri, 1990 for a discussion of host-preferred codon usage). Thus, the nucleotide sequences can be optimized for expression in any plant. It is recognized that all or any part of the gene sequence may be optimized or synthetic. That is, synthetic or partially optimized sequences may also be used. Variant nucleotide sequences and proteins also encompass sequences and protein derived from a mutagenic and recombinogenic procedure such as DNA shuffling. With such a procedure, one or more different coding sequences can be manipulated to create a new polypeptide possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art (see, for example, U.S. Pat. No. 5,605,794).

### Vectors

The disclosure further provides a vector comprising one or more nucleotide sequences described herein (e.g., a nucleotide sequence set forth in SEQ ID NO: 1 and/or SEQ ID NO: 2).

The term “vector” encompasses (but is not limited to) a phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector comprising the polynucleotides of described herein may comprise selectable markers for propagation and/or selection in a host. Further, the vector may be prepared from native (endogenous) and/or foreign (exogenous, heterologous) sequences with respect to the host.

Preferably, the vector referred to herein is suitable as a cloning vector, i.e. replicable in microbial systems. Such vectors ensure efficient cloning in bacteria, yeasts or fungi and make possible the stable transformation of plants. Examples include, e.g., various binary and co-integrated vector systems which are suitable for T DNA-mediated transformation. Such vector systems are generally characterized in that they contain at least the vir genes, which are required for the Agrobacterium-mediated transformation, and the sequences which delimit the T-DNA (T-DNA border). These vector systems also optionally comprise further cis-regulatory regions such as promoters and terminators and/or selection markers with which suitable transformed host cells or organisms can be identified. While co-integrated vector systems have vir genes and T DNA sequences arranged on the same vector, binary systems are based on at least two vectors, one of which bears vir genes, but no T-DNA, while a second one bears T DNA, but no vir gene. As a consequence, the last-mentioned vectors are relatively small, easy to manipulate and can be replicated both in E. coli and in Agrobacterium. An overview of binary vectors and their use can be found in Hellens et al, Trends in Plant Science (2000) 5, 446-451. Furthermore, by using appropriate cloning vectors, an expression cassette can be introduced into host cells or organisms such as plants or animals and, thus, be used in the transformation of plants, such as those which are published, and cited, in: Plant Molecular Biology and Biotechnology (CRC Press, Boca Raton, Florida), chapter 6/7, pp. 71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus, Annu. Rev. Plant Physiol. Plant Molec. Biol. 42 (1991), 205 225.

Suitable vector backbones are, in some embodiments, derived from vectors known in the art such as Okayama-Berg cDNA expression vector pcDV1 (Pharmacia), pCDM8, pRc/CMV, pcDNA1, pcDNA3 (Invitrogene) or pSPORT1 (GIBCO BRL). Further examples of typical fusion expression vectors are pGEX (Pharmacia Biotech Inc; Smith, D. B., and Johnson, K. S. (1988) Gene 67:31-40), pMAL (New England Biolabs, Beverly, MA) and pRIT5 (Pharmacia, Piscataway, NJ), where glutathione S-transferase (GST), maltose E-binding protein and protein A, respectively, are fused with the nucleic acid of interest encoding a protein to be expressed.

In some embodiments, the vector comprising one or more nucleotide sequences described herein is propagated and amplified in a plant cell. In some embodiments, one copy of the vector is propagated and amplified in a plant cell. In some embodiments, two or more (e.g., 3, 4, 5, 6 7, 8 or more) copies of the vector are propagated and amplified in a plant cell.

In some embodiments, the vector described herein comprises a promoter (e.g., a pericycle cell promoter) operably linked to a nucleotide sequence described herein (e.g., a nucleotide sequence set forth in SEQ ID NO: 1 and/or SEQ ID NO: 2). In some embodiments, the polynucleotide of interest is further operably linked to termination signals and/or other regulatory elements.

The term “promoter” as used herein refers to a nucleotide sequence, usually upstream (5′) to its coding sequence, which controls the expression of the coding sequence by providing the recognition site for RNA polymerase and other factors required for proper transcription. “Promoter” includes a minimal promoter that is a short DNA sequence comprised, in some cases, of a TATA box and other sequences that serve to specify the site of transcription initiation, to which regulatory elements are added for enhancement of expression. “Promoter” also refers to a nucleotide sequence that includes a minimal promoter plus regulatory elements and that is capable of controlling the expression of a coding sequence or functional RNA. This type of promoter sequence consists of proximal and more distal upstream elements, the latter elements often referred to as enhancers. Accordingly, an “enhancer” is a DNA sequence, which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements, derived from different promoters found in nature, or even be comprised of synthetic DNA segments.

The terms “operably linked” or “functionally linked” refer to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

In some embodiments, an operable linkage comprises a sequential arrangement of a nucleotide sequence encoding a promoter (e.g., a pericycle cell promoter), with a nucleic acid sequence to be expressed (such as a nucleotide sequence set forth in SEQ ID NO: 1 and/or SEQ ID NO: 2), and optionally, additional regulatory elements such as, for example, polyadenylation or transcription termination elements, enhancers, introns, etc, such that the nucleotide sequence of interest is expressed under the appropriate conditions (i.e., in a plant cell). Suitable arrangements include, e.g., those in which the nucleic acid sequence to be expressed is placed downstream (i.e., in 3′-direction) of the transcription regulating nucleotide sequence such that both sequences are covalently linked. Optionally, additional sequences may be inserted in-between the two sequences. Such sequences may be, for example, linker or multiple cloning sites. Furthermore, sequences can be inserted which encode parts of a fusion protein, in the event that a fusion protein comprising the product of the nucleic acid disclosed herein is desired. Preferably, the distance between the polynucleotide to be expressed and the transcription regulating nucleotide sequence is not more than 200 base pairs, such as not more than 100 base pairs or not more than 50 base pairs.

In some embodiments, the host cell is from a plant (e.g., a plant cell, a plant seed or other plant part). The term “plant” as used herein refers to a photosynthetic, eukaryotic multicellular organism. The term “plant parts” as used herein encompasses all components of a plant including seeds, shoots, stems, leaves, roots, flowers, and plant tissues and plant organs, plant cells, suspension cultures, callus tissue, embryos, meristematic regions, gametophytes, sporophytes, pollen, microspores and propagules. A “propagule” is any kind of organ, tissue, or cell of a plant capable of developing into a complete plant. A propagule can be based on vegetative reproduction (also known as vegetative propagation, vegetative multiplication, or vegetative cloning) or sexual reproduction. A propagule can therefore be a seed or part of the non-reproductive organs, like stem or leaf. In particular, with respect to Poaceae, suitable propagules can also be sections of the stem, i.e., stem cuttings.

A transgenic plant or plant part comprising a nucleotide vector described herein is specifically contemplated. An expression cassette or vector may be present in the cytoplasm of the organism or may be incorporated into the genome either by heterologous or by homologous recombination.

In some embodiments, the plant is a monocotyledonous plant, or the plant part is derived from a monocotyledonous plant. In some embodiments, the plant is a dicotyledonous plant, or the plant part is derived from a dicotyledonous plant.

In some embodiments, the plant (or plant part) is derived from the genera: Ananas, Musa, Vitis, Fragaria, Lotus, Medicago, Onobrychis, Trifolium, Trigonella, Vigna, Citrus, Carica, Persea, Prunus, Syragrus, Theobroma, Coffea, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Mangifera, Cichorium, Helianthus, Lactuca, Bromus, Asparagus, Antirrhinum, Heterocallis, Nemesia, Pelargonium, Panicum, Pennisetum, Ranunculus, Senecio, Salpiglossis, Cucurbita, Cucumis, Browaalia, Lolium, Malus, Apium, Gossypium, Vicia, Lathyrus, Lupinus, Pachyrhizus, Wisteria, Stizolobium, Agrostis, Phleum, Dactylis, Sorghum, Setaria, Zea, Oryza, Triticum, Secale, Avena, Hordeum, Saccharum, Poa, Festuca, Stenotaphrum, Cynodon, Coix, Olyreae, Phareae, Glycine, Pisum, Psidium, Passiflora, Cicer, Phaseolus, Lens, or Arachis.

In some embodiments, the plant (or plant part) is from the family of Poaceae, such as the genera Hordeum, Secale, Avena, Sorghum, Andropogon, Holcus, Panicum, Oryza, Zea, Triticum, for example the genera and species Hordeum vulgare, Hordeum jubatum, Hordeum murinum, Hordeum secalinum, Hordeum distichon, Hordeum aegiceras, Hordeum hexastichon, Hordeum hexastichum, Hordeum irregulare, Hordeum sativum, Hordeum secalinum, Secale cereale, Avena sativa, Avena fatua, Avena byzantina, Avena fatua var. sativa, Avena hybrida, Sorghum bicolor, Sorghum halepense, Sorghum saccharatum, Sorghum vulgare, Andropogon drummondii, Holcus bicolor, Holcus Sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum, Panicum militaceum, Oryza sativa, Oryza latifolia, Zea mays, Triticum aestivum, Triticum durum, Triticum turgidum, Triticum hybernum, Triticum macha, Triticum sativum or Triticum vulgare.

In some embodiments, the plant is a non-legume plant. In some embodiments, the non-legume plant is a rice plant, a barley plant, a maize plant, an oat plant, a rye plant, a Sorghum plant, a wheat plant or a Poaceae grass plant.

### Methods of Producing a Transgenic Plant

The disclosure also provides a method for producing a non-legume plant capable of spontaneous nodule formation comprising introducing one or more nucleotide sequences described herein (e.g., a nucleotide sequence at least 95% identical to SEQ ID NO: 1 or SEQ ID NO: 2) into the genome of the plant. Methods of stimulating spontaneous nodule formation in a non-legume plant comprising introducing one or more nucleotide sequences that are at least 85% identical to SEQ ID NO: 1 or SEQ ID NO: 2 into the genome of the plant are also contemplated. In some embodiments, the nucleotide sequence is operably linked to a pericycle cell promoter.

A variety of techniques are available and known to those skilled in the art for introduction of constructs (e.g., vectors) into a host cell (e.g., plant cell). Exemplary techniques include transformation with DNA employing A. tumefaciens or A. rhizogenes as the transforming agent, liposomes, PEG precipitation, electroporation, DNA injection, direct DNA uptake, microprojectile bombardment, particle acceleration, and the like (See, for example, EP 295959 and EP 138341). However, cells other than plant cells may be transformed with the vector described herein. The general descriptions of plant expression vectors and reporter genes, and Agrobacterium and Agrobacterium-mediated gene transfer, can be found in Gruber et al. (1993).

Other transformation methods are available to those skilled in the art, such as direct uptake of foreign DNA constructs (see EP 295959), techniques of electroporation (Fromm 1986) or high velocity ballistic bombardment with metal particles coated with the nucleic acid constructs (e.g., U.S. Pat. No. 4,945,050). Once transformed, the cells can be regenerated by those skilled in the art. Those skilled in the art will appreciate that the choice of method might depend on the type of plant, i.e., monocotyledonous or dicotyledonous, targeted for transformation.

In some embodiments, the methods described herein comprise introducing one or more vectors comprising the one or more nucleotide sequences into the plant by transformation. In some embodiments, the methods described herein comprise introducing a vector comprising the nucleotide sequence set forth in SEQ ID NO: 1 into the plant by transformation. In some embodiments, the methods described herein comprise introducing a vector comprising the nucleotide sequence set forth in SEQ ID NO: 2 into the plant by transformation. In some embodiments, the methods described herein comprise introducing a first vector comprising the nucleotide sequence set forth in SEQ ID NO: 1 and a second vector comprising the nucleotide sequence set forth in SEQ ID NO: 2 into the plant by transformation. In some embodiments, the methods described herein comprise introducing a vector comprising both the nucleotide sequence set forth in SEQ ID NO: 1 and the nucleotide sequence set forth in SEQ ID NO: 2 into the plant by transformation.

If desired, the vector may comprise a selectable marker, which may provide resistance to an antibiotic (e.g., kanamycin, hygromycin or methotrexate) or a herbicide (e.g., phosphinothricin), or a separate vector encoding a selectable marker may be utilized in conjunction with the vector comprising the nucleotide of interest described above. For certain plant species, different antibiotic or herbicide selection markers may be preferred. Selection markers used routinely in transformation include the nptII gene which confers resistance to kanamycin and related antibiotics, the bar gene which confers resistance to the herbicide phosphinothricin, the hph gene which confers resistance to the antibiotic hygromycin, and the dhfr gene which confers resistance to methotrexate.

Methods for the production and further characterization of stably transformed plants are well-known to the person skilled in the art. As an example, transgenic plant cells are placed in an appropriate selective medium for selection of transgenic cells, which are then grown to callus. Shoots are grown from callus. Plantlets are generated from the shoot by growing in rooting medium. When a selection marker is used, the marker allows for selection of transformed cells as compared to cells lacking the DNA.

The term “expression” as used herein refers to the transcription and/or translation of nucleic acid (e.g., transgene) in a plant cell. The expression of a polynucleotide of interest in a host cell (e.g., a plant cell) can be determined by various well known techniques, e.g., by Northern Blot or in situ hybridization techniques as described in WO 02/102970, the disclosure of which is incorporated by reference in its entirety.

To confirm the presence of the transferred polynucleotide of interest in transgenic cells and plants, a variety of assays may be performed. The expression of a polynucleotide of interest in a host cell (e.g., a plant cell) can be determined by various well known techniques, e.g., by Northern Blot or in situ hybridization techniques as described in WO 02/102970, the disclosure of which is incorporated by reference in its entirety. Such assays include, Northern Blot or in situ hybridization techniques as described in WO 02/102970, in situ hybridization and nucleic acid-based amplification methods such as PCR or RT-PCR or TaqMan; immunological assays such as ELISAs and Western blots, and also, by analyzing the phenotype of the whole regenerated plant (e.g., presence of nodule formation in a non-nodulating plant).

In some embodiments, the methods described herein comprise sexually crossing a plant with the transgenic plant described herein.

In some embodiments, the methods described herein further comprise contacting the plant with a bacteria that can form nodules on plants of the Fabale (legumes), Cucurbitale, Fagale and Rosale families. This includes but is not limited to bacteria from the genera Frankia, Ensifer, Azorhizobium, Agrobacterium, Rhizobium, Allorhizobium, Neorhizobium, Bradyrhizobium, Sinorhizobium, Mesorhizobium, Cupriavidus, or Burkholderia.

In some embodiments, the methods described herein occur in the absence of cytokinin.

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth.

### EXAMPLE

### Materials and Methods

Plant material: Seeds from the Medicago truncatula genotype A17 were scarified 8 minutes in sulfuric acid and sterilized for 4 min in bleach. After washing with sterilized water, seeds were sown on 1% agar plates with 1 μM of GA₃ and stored for 3 days at 4° C. before incubating overnight at 24° C. in the dark. Germinated seedlings were transferred to square plates containing filter paper-lined modified Fahraeus medium or buffer nodulation medium (BNM) supplemented with 0.1 μM aminoethoxyvinylglycine (AVG; Sigma-Aldrich Company Ltd, Darmstadt, Germany) and grown vertically in chambers at 24° C. under long-day conditions (16 h light at 150 μmol m⁻² s⁻¹ light intensity/8 h dark).

Nuclei isolation from Medicago roots for single nuclei RNA-seq: After 24 hours of E. meliloti treatment, 0.5-0.7-cm long root portions around the drop-inoculated spot were cut from 150 2-day-old M. truncatula seedlings. Root portions were placed on a precooled glass plate on the top of an aluminum heating/cooling block kept on ice with 200 μL of NIB1 1× supplemented with 0.5 U/μL Protector RNase Inhibitor. Once all root segments were collected, they were chopped with a razor blade for 2 min. This step was repeated twice with a 30 sec interval in between. Then, the sample was transferred to a 50 mL Falcon tube and nuclei were isolated. After the last wash step, the pellet was gently resuspended in a final volume of 750 μL of NIB2 1× containing 0.2 U/μL Protector RNase Inhibitor using a Rainin 1 mL wide-bore tip and transferred to a 5 mL sterile tube compatible with the cell sorter. Nuclei were stained with 5 μg/ml DAPI for 5 min at room temperature and sorted using a BD FACSAria™ IIU/III upgraded cell sorter.

Single nuclei cDNA and library preparation: 20k nuclei were used to generate each single-nuclei RNA-seq (snRNA-seq) library following the 10× Genomics Chromium Single Cell v3.1 protocol. Two snRNA-seq libraries from two independent spot-inoculation experiments were generated.

Cell clustering: Integration of the replicates and clustering was performed using Asc-Seurat. Re-clustering of the pericycle cells was performed using the following parameters: N of variable genes=3000; PCs=40; resolution=0.2.

Cloning: A 153 bp and a 102 bp fragment of the MtHK1 and LRP1 mRNA, set forth in SEQ ID NOs: 1 and 2 respectively, was amplified by PCR from M. truncatula A17 root cDNA and cloned into pENTR Directional TOPO vector following the manufacturer's instructions (Invitrogen, Carlsbad, California), to generate the Gateway entry clone vector. To generate the expression vector, the LR reaction was performed with the entry clone vector and the pK7GW1GW2 (II)-RedRoot destination vector (gatewayvectors.vib.be/collection/pk7gwiwg2ii-redroot), using the LR clonase kit (Invitrogen, Carlsbad, California).

Generation of composite plants and nodulation assay: Composite M. truncatula A17 plants were generated by inoculating one-day-old seedlings with Agrobacterium rhizogenes harboring the tdTomato selection marker described above and the MtHK1 and LRP1 fragments. Three weeks after transformation with A. rhizogenes MSU440, the roots were screened for the red fluorescence of DsRED and the composite plants with red roots were transferred to growth pouches (mega-international.com/tech-info/) containing modified nodulation medium (MNM). The plants were acclimated for a week and then each pouch was inoculated with 1 ml of E. meliloti 1021 (OD600=0.1) harboring the pXLGD4 and expressing lacZ under the hemA promoter (Leong et al. 1985). The MNM was replenished 3 days after transferring the plants, and every week thereafter. Two weeks after inoculation, the total number of nodules per plant was scored as the number of nodules present in all the fluorescence roots in each plant, under an Olympus MVX10 fluorescence stereo microscope. Then, live seedlings were stained for lacZ activity evaluation (5 mM potassium ferrocyanide, 5 mM potassium ferricyanide, and 0.08% X-gal in 0.1 M PIPES, pH 7) overnight at 37° C. Roots were rinsed in distilled water and the presence of the bacteria inside the nodules of the transgenic roots was also reported by observing the X-gal hydrolyzation (blue product).

Nodule development induction in Populus: GoldenGate technology was used to assemble multi-gene constructs and express candidate genes as previously described (Patron et al., 2015). MtHK1 and LRP1 were expressed both individually and in combinations. Each gene was driven by a pericycle- or a cortex-specific promoter. Constructs were transformed into poplar roots using Agrobacterium rhizogenes-mediated transformation. After transformed roots emerged, the roots were evaluated for the presence of nodule structures.

### Results and Discussion

Nuclei from complex plant tissues were isolated as described previously (Conde et al. 2021), to dissect the transcriptome of M. truncatula root individual cells responding to rhizobial inoculation. To characterize the early transcriptome reprogramming of root cells responding to rhizobia, nuclei were isolated from 150 M. truncatula root infections zone segments in each of two replicated experiments, 24 hours after spot inoculation with E. meliloti. High-throughput, microfluidic-based single-nuclei RNA sequencing (snRNA-seq) was performed using the 10× Genomics Chromium technology, as described previously (Conde et al. 2021). 18,016 nuclei were captured across two independent biological replicates and obtained on average 68,270 reads per nucleus. A median of 464 unique molecular identifiers (UMIs), corresponding to the expression of a median of 399 genes, were detected per nucleus. Overall, 28,748 genes were detected among the two biological replicates. To eliminate potentially empty gel bead-in emulsions (GEMs), which contain only ambient RNA, the data at both nuclei and UMI levels was filtered. This step resulted in a final count of 3,351 and 4,855 nuclei, a mean of 640 and 838 expressed genes per nucleus, and the expression of a total of 25,278 and 27,709 genes in replicates 1 and 2, respectively.

To identify the distinct cell populations contained in the M. truncatula root sections, cell clustering was performed after integrating the single-cell transcriptomes obtained in both biological replicates, using Seurat (Stuart et al. 2019; Hao et al. 2021), implemented on Asc-Seurat (Pereira et al. 2021). The integrated dataset comprises 8,234 nuclei, and 24,306 expressed genes. After the integration, these clusters harbored similar numbers of cells from each replicate, and their gene expression profiles were highly correlated between replicates (r>0.95), highlighting the reproducibility of the snRNA-seq procedure.

To attribute cell identities to each cluster, the expression profile of M. truncatula homologs of 187 cell-type-specific markers previously identified in Arabidopsis roots was explored (Ryu et al. 2019; Zhang et al. 2019b; Denyer et al. 2019; Shulse et al. 2019). A cluster lacking enrichment for any known markers and two clusters containing fewer than 1% of the total number of nuclei detected was excluded from further characterization. In total, 4,000 nuclei were distributed in seven clusters based on their transcriptome profiles, with each cluster containing between 166 to 1263 nuclei. Based on previously known cell-type-specific markers, nuclei derived from six types of cells was identified: root hair (clusters 1), epidermis (cluster 4), cortex (cluster 5), endodermis (cluster 2), pericycle (cluster 3), and stele (excluding pericycle) (clusters 0 and 6).

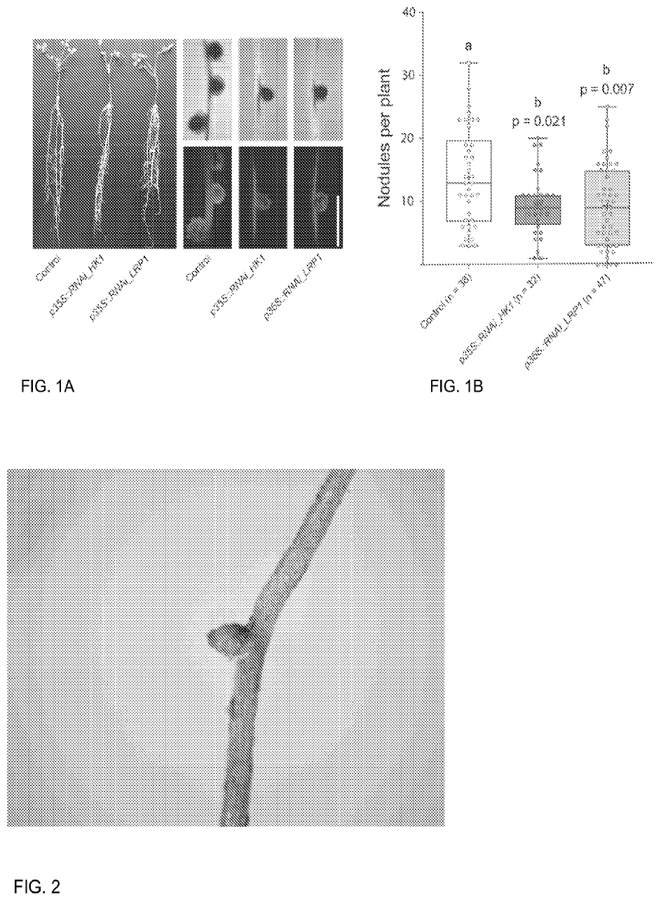

A set of 140 M. truncatula homologs of Arabidopsis marker genes for the stele (procambium/vascular cambium, phloem, xylem, and pericycle) were enriched in clusters 0, 3, and 6. Cluster 0 is characterized by the expression of markers for most of the stele cell types, including phloem (e.g., M. truncatula homolog of Arabidopsis CALLOSE SYNTHASE 7 (Xie et al. 2011), MtrunA17Chr3g0096261), xylem (e.g., homolog of Arabidopsis ACAULIS 5 (Muñiz et al. 2008), MtrunA17Chr5g0394371), and genes involved in maintaining vascular cell organization (e.g., homologs of Arabidopsis REVOLUTA (S et al. 2013), MtrunA17Chr2g0326731 and MtrunA17Chr4g0028991) (FIGS. 1A and 1B). Markers for early cell proliferation of the vascular procambial/cambial cells in the stele (e.g., homologs of Arabidopsis CYCLIN A1;1 (Zhang et al. 2019a), MtrunA17Chr3g012505) were detected in a sub-cluster of the stele related to cell division (cluster 6). In contrast, cluster 3 is highly distinct transcriptionally and characterized by the expression of a subset of markers for the pericycle, a cell-type composed of a unique layer, including homologs of Arabidopsis NITRATE TRANSPORTER 1.5 genes (Lin et al. 2008) (NTR1.5, MtrunA17Chr5g0400371, MtrunA17Chr4g0054891).

The pericycle is a cell type of primary interest in symbiotic nitrogen fixation because of its early mitotic activation after root inoculation with rhizobia. After inoculation with E. meliloti, the early activation of cell division in M. truncatula roots is limited to few cells of the pericycle (Xiao et al. 2014). Cells identified previously as representing the pericycle were re-clustered at a higher clustering resolution, separating them into two sub-clusters (Cluster 3-0:372, Cluster3-1:118 cells). Enrichment for transcription of genes previously shown to be induced by E. meliloti was assessed (Schiessl et al. 2019) in these two sub-clusters. It was observed that genes that were highly upregulated in spot inoculated M. truncatula roots, within 12 to 36 hours post treatment (log fold-change≥1.5, p-value adjusted≤0.05, and expressed in at least 5% of the cells in any of the pericycle's clusters) were detected primarily, and at higher expression in cells of the cluster 3-1. In combination, this data suggests that cluster 3-1 represents cells undergoing the initial transcriptional changes related to early nodule formation.

As the pericycle cells respond to the presence of rhizobia by transitioning from a steady to a mitotically activated state, they follow a continuous, gradual transcriptional change that later results in the cells at the base of a newly formed nodule. Pseudotime analysis was used to reconstruct the temporal transcriptional changes in this cell lineage (Street et al. 2018), assigning starting (cluster 3-0) and terminal (cluster 3-1) clusters. A Random Forest regression (Saelens et al. 2019) algorithm was applied to rank genes according to their importance in predicting cells' positions in the trajectory and a generalized additive model regression (Van den Berge et al. 2020) to detect genes significantly associated in their expression with the inferred trajectory. We compared the most predictive genes of both methods and detected a homolog of LATERAL ROOT PRIMORDIUM 1 (LRP1, MtrunA17Chr3g0082511), a member of the SHORT INTERNODES/STYLISH (SHI/STY) family, involved in auxin signaling during lateral root (S et al. 2020) and nodule (Schiessl et al. 2019) formation; and MtHK1 (MtrunA17Chr5g0405701), a histidine kinase.

The significant association between the expression of MtLRP1 and MtHK1 to the differentiation of pericycle cells responding to rhizobia, and their possible role in auxin and cytokinin signaling, suggest a function in early nodule development. To test whether MtLRP1 and MtHK1 are involved in nodulation, RNA-mediated interference (RNAi) was used to downregulate the expression of MtLRP1 and MtHK1. Composite M. truncatula plants were generated expressing the RNAi constructs or not (empty vector), and the plants were inoculated with E. meliloti harboring phemA: lacZ (Leong et al. 1985), which was used to monitor successful nodule infection (FIG. 1, left panel). Two weeks post inoculation, a significant decrease in the number of nodules was observed in the roots expressing the RNAi construct targeting MtLRP1 and MtHK1 in comparison with the roots transformed with the empty vector (FIG. 1, right panel), demonstrating that LRP1 and HK1 are required for nodule organogenesis.

Next, whether MtLRP1 and/or MtHK1 are sufficient to induce the formation of nodule structure in Populus, individually or in combination, was assessed. Following genetic transformation with A. rhizogenes, under control of a pericycle-specific promoter, the transformed roots were assessed for the presence of nodules when grown in media with or without cytokinin. Roots transformed with MtLRP1 showed nodule structures when grown in media with cytokinin, but these structures were not detected in the absence of the phytohormone. In contrast, roots transformed with MtHK1 under control of the pericycle promoter, or together with MtLRP1 under the same promoter, resulted in the presence of nodules, even without phytohormones in the media (FIG. 2).

### REFERENCES

Conde et al., PLOS One 16, 2021
Denyer et al., Dev Cell 48:840-852.e5, 2019
Hao et al., Cell 184:3573-3587.e29, 2021.
Leong et al., Nucleic Acids Res 13:5965-5976, 1985.
Lin et al., Plant Cell 20:2514-2528, 2008.
Muñiz et al., Development 135:2573-2582, 2008.
P T, H W S, C D, M K, D C. 2021. Simple, efficient and open-source CRISPR/Cas9 strategy for multi-site genome editing in Populus tremula×alba. Tree Physiol. www.pubmed.ncbi.nlm.nih.gov/33960379/.
Pereira W J, Almeida F, Balmant K, Rodriguez D, Triozzzi P, Schmidt H, Dervinis C, Pappas Jr G, Kirst M. 2021. Asc-Seurat-Analytical single-cell Seurat-based web application. bioRxiv 2021.03.19.436196. www.//doi.org/10.1101/2021.03.19.436196.
Ryu et al., Plant Physiol 179:1444-1456, 2019.
S M, et al., EMBO J 32:178-193, 2013.
S S et al., Plant J 101:87-100, 2020.
Saelens et al., Nat Biotechnol 37:547-554, 2019.
Schiessl et al., Curr Biol 29:3657-3668.e5, 2019.
Shulse et al., Cell Rep 27:2241-2247.e4, 2019.
Street et al., BMC Genomics 19:477, 2018.
Stuart et al., Cell 177:1888-1902.e21, 2019.
Van den Berge et al., Nat Commun 11:1-13, 2020.
Xiao et al., Development 141:3517-3528, 2014.
Xie et al., Plant J 65:1-14, 2011.
Zhang et al., Nat Plants 510 (5): 1033-1042, 2019.
Zhang et al., Mol Plant 12:648-660, 2019

What is claimed is:

1. A transgenic plant comprising one or more nucleotide sequences that is at least 85% identical to SEQ ID NO: 1 or SEQ ID NO: 2.

2. The transgenic plant of claim 1, wherein the nucleotide sequence comprises the nucleotide sequence of SEQ ID NO: 1.

3. The transgenic plant of claim 1, wherein the nucleotide sequence comprises the nucleotide sequence of SEQ ID NO: 2.

4. The transgenic plant of any one of claims 1-3, comprising nucleotide sequences set forth in SEQ ID NO: 1 and SEQ ID NO: 2.

5. The transgenic plant of any one of claims 1-4, wherein the nucleotide sequence is operably linked to a pericycle cell promoter.

6. The transgenic plant of any one of claims 1-5, wherein the one or more nucleotide sequences is expressed in a pericycle cell of the plant.

7. The transgenic plant of any one of claims 1-5, wherein the one or more nucleotide sequences is expressed in a cortex cell of the plant.

8. The transgenic plant of any one of claims 1-7, wherein the plant is a monocotyledonous plant.

9. The transgenic plant of any one of claims 1-8, wherein the plant is a dicotyledonous plant.

10. The transgenic plant of any one of claims 1-9, wherein the plant is a non-legume plant.

11. The transgenic plant of claim 10, wherein the non-legume plant is a rice plant, a barley plant, a maize plant, an oat plant, a rye plant, a Sorghum plant, a wheat plant or a Poaceae grass plant.

12. A method of producing a non-legume plant capable of spontaneous nodule formation comprising introducing one or more nucleotide sequences that are at least 85% identical to SEQ ID NO: 1 or SEQ ID NO: 2 into a cell of the plant.

13. The method of claim 12, wherein the nucleotide sequence is operably linked to a pericycle cell promoter.

14. The method of claim 12 or claim 13, wherein the cell is a pericycle cell.

15. The method of claim 12 or claim 13, wherein the cell is a cortex cell.

16. The method of any one of claims 12-15, comprising introducing the one or more vectors comprising the nucleotide sequences into the plant by transformation.

17. The method of any one of claims 12-16, comprising sexually crossing a plant with the transgenic plant set forth in any one of claims 1-11.

18. The method of any one of claims 12-17, wherein the method occurs in the absence of cytokinin.

19. A method of stimulating spontaneous nodule formation in a non-legume plant comprising introducing one or more nucleotide sequences that are at least 85% identical to SEQ ID NO: 1 or SEQ ID NO: 2 into a cell of the plant.

20. The method of claim 19, comprising introducing a nucleotide sequence set forth in SEQ ID NO: 1 into a cell of the plant.

21. The method of claim 19, comprising introducing a nucleotide sequence set forth in SEQ ID NO: 2 into a cell of the plant.

22. The method of claim 19, comprising introducing the nucleotide sequence set forth in SEQ ID NO: 1 and the nucleotide sequence set forth in SEQ ID NO: 2 into a cell of the plant.

23. The method of claim 19, wherein the nucleotide sequence set forth in SEQ ID NO: 1 and the nucleotide sequence set forth in SEQ ID NO: 2 are provided in a single vector.

24. The method of claim 19, wherein the nucleotide sequence set forth in SEQ ID NO: 1 and the nucleotide sequence set forth in SEQ ID NO: 2 are provided in separate vectors.

25. The method of any one of claims 19-24, wherein the nucleotide sequence is operably linked to a pericycle cell promoter.

27. The method of any one of claims 19-24, wherein the cell is a pericycle cell.

28. The method of any one of claims 19-24, wherein the cell is a cortex cell.

29. The method of any one of claims 19-24, comprising introducing the one or more nucleotide sequences into the plant by transformation.

30. The method of any one of claims 19-28 further comprising sexually crossing a plant with the transgenic plant set forth in any one of claims 1-11.

31. The method of any one of claims 19-30, further comprising contacting the plant with a bacteria from the genus Rhizobium, Bradyrhizobium, Sinorhizobium, or Mesorhizobium.

32. The method of claim 31, wherein the bacteria is E. meliloti.

33. The method of any one of claims 19-32, wherein the method occurs in the absence of cytokinin.

34. A seed produced by the plant of any one of claims 1-11.
